(19) United States
(12) Patent Application Publication
(10) Pub. No.: US 2024/0114928 A1
(43) Pub. Date: Apr. 11, 2024

(54) MINERAL NUTRITIONAL SUPPLEMENTS FOR PET FOOD TO REDUCE FUR LOSS AND IMPROVE COAT SHINE

(71) Applicant: Zinpro Corporation, Eden Prairie, MN (US)

(72) Inventors: Dana Tomlinson, Eden Prairie, MN (US); Erik Swensson, Eden Prairie, MN (US); Brent Kirn, Eden Prairie, MN (US)

(21) Appl. No.: 18/472,525

(22) Filed: Sep. 22, 2023

Related U.S. Application Data
(60) Provisional application No. 63/376,670, filed on Sep. 22, 2022.

(51) Int. Cl. A23K 20/20; A23K 20/142; A23K 50/20; A23K 50/42; A61K 31/198; A61K 33/26; A61K 33/30; A61K 33/32; A61K 33/34; A61P 17/00

(52) U.S. Cl. CPC A23K 20/30; A23K 20/142; A23K 50/20; A23K 50/42; A61K 31/198; A61K 33/26; A61K 33/30; A61K 33/32; A61K 33/34; A61P 17/00

(57) ABSTRACT

Methods of nutritionally supplementing the diet of an animal with trace mineral mixed amino acid complexes are provided herein. Also described are methods of reducing shedding and improving coat color and shine in a canine or feline. Methods of manufacturing a pet food comprising trace mineral mixed amino acid complexes are also described.

### CROSS REFERENCE TO RELATED APPLICATIONS

This application is claims priority under 35 U.S.C. § 119 to provisional patent application U.S. Ser. No. 63/376,670, filed Sep. 22, 2022, which is incorporated by reference in its entirety, including without limitation, the specification, claims, and abstract, as well as the figures and tables therein.

### TECHNICAL FIELD

The present disclosure relates generally to animal nutritional supplementation of trace minerals in a highly bioavailable, absorbable format.

### BACKGROUND

Trace minerals are essential to maintain healthy skin, coat, and a pet's overall wellbeing. Of these minerals, zinc, manganese, copper, iron, iodine, and selenium all play significant roles in health maintenance processes. For example, Zinc plays a role in over 300 enzymes and is a component in over 3000 proteins responsible for structural, catalytic, and regulatory functions within the mammalian body. It is through these diverse physiological processes and metabolic functions that trace minerals affect the immune system, endocrine system, skin integument, production of healthy hair, wound healing, and overall health.

Trace mineral insufficiency is often characterized by various clinical signs such as skin lesions, slow and sparse hair growth, conjunctivitis, emaciation, poor growth, abnormal sexual development, keratitis, digestive upset, and vomiting. Ear and skin infections and allergic skin issues, such as hot spots, are among the top issues sparking veterinary visits for companion animals. Management of these conditions is costly and often requires numerous follow-up appointments. Unfortunately, it has been found that even pets fed well-balanced commercial foods containing sufficient quantities of trace minerals can show signs of trace mineral insufficiency. Zinc and other trace mineral absorption is often hindered by certain nutrients or food ingredients commonly found in commercial pet foods, leading to reduced bioavailability of the trace minerals. Improving trace mineral bioavailability can result in improvements in skin integrity, immune competence, hair length and density, and enhanced animal wellbeing.

Accordingly, there exists a need in the art for trace mineral nutritional supplements with improved bioavailability that can be fed as a supplement or incorporated into pet food formulations.

### SUMMARY

Methods of nutritionally supplementing the diet of a canine or feline are provided. In some embodiments, the method comprises feeding the canine or feline a mineral supplement comprising a copper mixed amino acid complex, an iron mixed amino acid complex, a manganese mixed amino acid complex, and a zinc mixed amino acid complex. In some embodiments, the zinc complex is in an amount between about 6 wt. % and about 10 wt. %, the iron complex is in an amount between about 2.5 wt. % and about 5 wt. %, the manganese complex is in a concentration of between about 1 wt. % and about 4 wt. %, and the copper complex is in a concentration of between about 0.2 wt. % and about 2 wt. %.

Methods of reducing shedding in a canine or feline are also provided. In some embodiments, the method comprises feeding the canine or feline a mineral supplement comprising a copper mixed amino acid complex, an iron mixed amino acid complex, a manganese mixed amino acid complex, and a zinc mixed amino acid complex. In some embodiments, the zinc complex is in an amount between about 6 wt. % and about 10 wt. %, the iron complex is in an amount between about 2.5 wt. % and about 5 wt. %, the manganese complex is in a concentration of between about 1 wt. % and about 4 wt. %, and the copper complex is in a concentration of between about 0.2 wt. % and about 2 wt. %.

Also provided are methods of manufacturing a pet food for a canine or a feline. In some embodiments, the method comprises adding a mineral supplement to the pet food, wherein the mineral supplement comprises a copper mixed amino acid complex, an iron mixed amino acid complex, a manganese mixed amino acid complex, and a zinc mixed amino acid complex.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Several embodiments in which the present disclosure can be practiced are illustrated and described in detail. As such, the figures and examples are non-limiting.

FIG. 1 is a photograph showing an example of the hair growth point of evaluation on a dog.

FIG. 2 is a photograph showing an example of the hair growth point of evaluation on a dog.

FIG. 3A is a line graph comparing hair growth over time with different mineral supplement compositions.

FIG. 3B is a line graph comparing hair growth over time with different mineral supplement compositions.

FIG. 3C is a bar graph comparing hair growth over time with different mineral supplement compositions.

FIG. 4 is a bar graph comparing hair growth over time with different mineral supplement compositions in different dog breeds.

FIG. 5A is a bar graph examining hair growth over time in different dog breeds.

FIG. 5B is a bar graph comparing hair growth over time with different mineral supplement compositions in different dog breeds.

FIG. 6A provides two color photographs of a dog on the Control diet before and after the dietary supplementation (Left, the dog prior to shaving; Right, the same dog at end of study period); the color images demonstrate the coat color and sheen before and after treatment for comparison.

FIG. 6B provides two color photographs of a dog on the Control diet before and after the dietary supplementation (Left, the dog prior to shaving; Right, the same dog at end of study period); the color images demonstrate the coat color and sheen before and after treatment for comparison.

FIG. 7A is a line graph comparing hair shedding over time with different mineral supplement compositions.

FIG. 7B is a bar graph comparing hair shedding over time with different mineral supplement compositions.

FIG. 8 is a provides two color photographs of a dog before and after treatment with Treatment 1, where shedding can be observed as well as improvement in hair color and sheen.

FIG. 9A provides two color photographs of a dog on the Treatment 1 diet before and after treatment (Left, the dog prior to shaving; Right, the same dog at end of study period); the breed corresponds to that of FIG. 6A and the color images demonstrate the coat color and sheen before and after treatment for comparison.

FIG. 9B provides two color photographs of a dog on the Treatment 1 diet before and after treatment (Left, the dog prior to shaving; Right, the same dog at end of study period); the breed corresponds to that of FIG. 6B and the color images demonstrate the coat color and sheen before and after treatment for comparison.

FIG. 10A is a color photograph of a dog's paw in before treatment with the control; the coloration and skin health can be observed via color photograph.

FIG. 10B is a color photograph of a dog's paw in before treatment with the Treatment 1; the coloration and skin health can be observed via color photograph.

FIG. 10C is a color photograph of a dog's paw in after treatment with the control; the coloration and skin health can be observed via color photograph.

FIG. 10D is a color photograph of a dog's paw in after treatment with the Treatment 1; the coloration and skin health can be observed via color photograph.

FIG. 11 is a line graph comparing heel tissue thickness over time with different mineral supplement compositions.

FIG. 12 is a line graph comparing abdominal tissue thickness over time with different mineral supplement compositions.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Biological, chemical, process, and/or other changes can be made without departing from the spirit and scope of the present disclosure. No features shown or described are essential to permit basic operation of the present disclosure unless otherwise indicated.

The terminology used herein is for the purpose of describing particular embodiments only and it is not intended to be limiting in any manner or scope. For example, as used in this specification and the appended claims, the singular forms “a”, “an”, and “the” can include plural referents unless the content clearly indicates otherwise. Further, all units, prefixes, and symbols may be denoted in its SI accepted form.

Numeric ranges recited within the specification are inclusive of the numbers within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range (e.g., 1 to 5 includes 1, 1.5, 2, 2¾, 3, 3.80, 4, and 5).

Terms characterizing sequential order, a position, and/or an orientation are not limiting and are only referenced according to the views presented.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation. The preferred materials and methods are described herein. In describing and claiming the embodiments of the present disclosure, the following terminology will be used in accordance with the definitions set out below.

As used herein, the term “about,” refers to variation in the numerical quantity that can occur, for example, through typical measuring techniques and equipment, with respect to any quantifiable variable, including, but not limited to, mass, volume, time, length, diameter, percent, reflectance, and concentration. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “animal” refers to any animal that could benefit from supplementation with trace minerals, e.g., a human, bovine, canine, equine, feline, hircine, lupine, murine, ovine, and porcine animals.

The term “pet” or “companion animal” refers to domesticated animals such as cats, dogs, rabbits, guinea pigs, ferrets, hamsters, mice, gerbils, horses, cows, goats, sheep, donkeys, pigs, and the like. Animals of all ages are included, e.g., young animals, adults, animals of medium age and seniors. Of primary interest with respect to the present disclosure are dogs and cats, at all shapes and body constitution (normal weight, overweight, obese).

The term “pet food” refers to a composition that is intended for ingestion by a companion animal. As contemplated herein, the pet food of the present disclosure is meant to encompass nutritionally complete and balanced animal food compositions that additionally comprise high bioavailability trace minerals. The methods of this disclosure utilize compositions that are not intended to be restricted by any specific listing of proteinaceous or fat ingredients or product form. The compositions can be prepared in, for example, a dry, kibble, canned, wet, or intermediate moisture form using conventional pet food processes. For equine animals, the “pet food” includes distillers' fermentation solubles, feed grains, corn cob flour, hay, rice, flaxseed, soybeans, soybean stalks, whey, and other cellulosic feed materials.

The term “kibble” refers to a dry, ground, processed pet food composition shaped into small pellets or shapes.

A “nutritionally complete diet” is a diet that includes sufficient nutrients for maintenance of normal health of a healthy animal on the diet. Nutritionally complete and balanced pet food compositions are familiar to one of skill in the art. For example, substances such as nutrients and ingredients suitable for nutritionally complete and balanced animal feed compositions, and recommended amounts thereof, may be found for example, in the Official Publication of The Association of American Feed Control Officials, Inc. (AAFCO), Champaign, IL, 2022, or the National Research Council's Nutrient Requirements of Dogs and Cats, The National Academy Press, Washington, D.C., 2006.

A used herein, the term “supplement(s)” include but are not limited to, a feed used with another feed to improve nutritive balance or performance of the total. Supplements include but are not limited to, compositions that are fed undiluted as a supplement to other feeds, offered free choice with other parts of an animal's ration that are separately available, or diluted and mixed with an animal's regular feed to produce a complete feed. The AAFCO guidelines, for example, contain a discussion relating to supplements in the Official Publication of The Association of American Feed Control Officials, Inc. (AAFCO), Champaign, IL, 2022, or the National Research Council's Nutrient Requirements of Dogs and Cats, The National Academy Press, Washington, D.C., 2006.

Supplements may be in various forms including, but not limited to, powders, liquids, syrups, pills, encapsulated compositions, and the like.

### Mineral Supplements

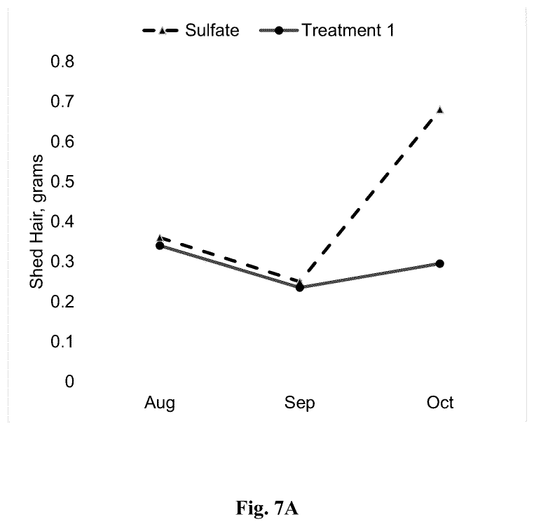

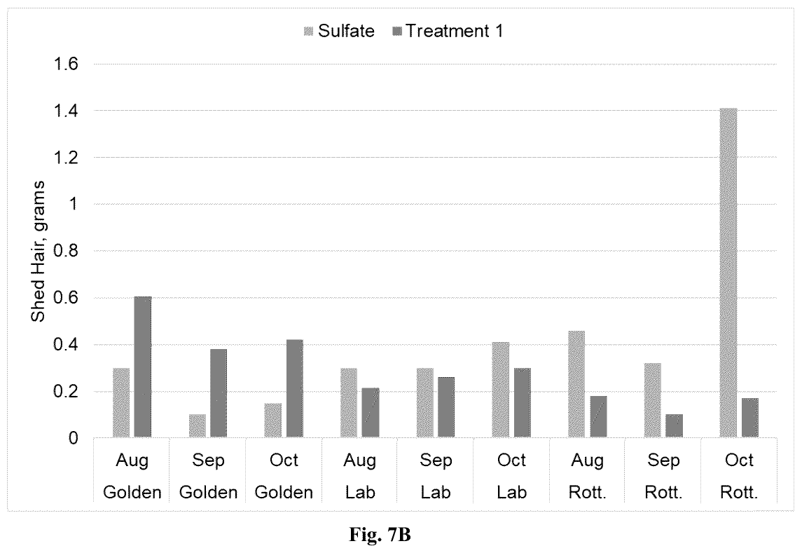

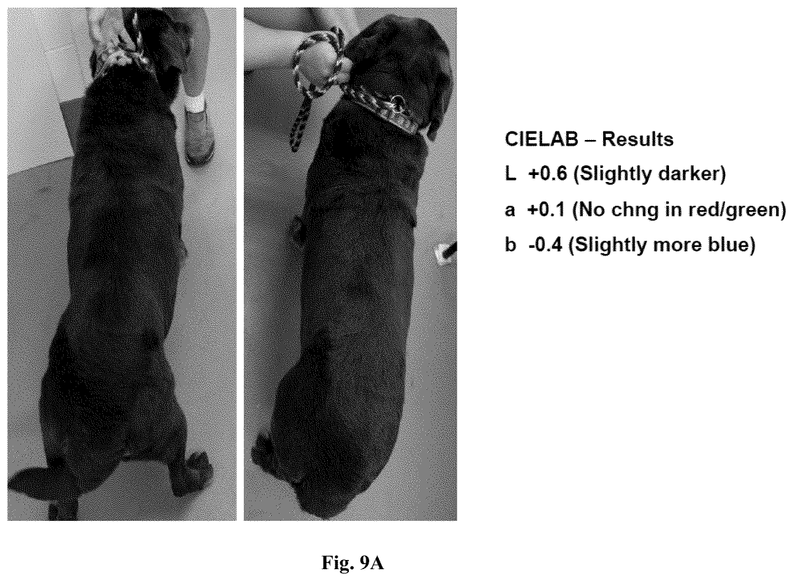

Provided herein are trace mineral supplements and methods related to same. The trace mineral supplements of the present disclosure have improved bioavailability due to their association with an organic molecule ligand as compared to trace minerals in their inorganic form. Nutritionally supplementing the diet of an animal with trace mineral supplements with improved bioavailability profiles, as disclosed herein, has numerous advantages. For example, an animal fed the disclosed trace mineral supplements may exhibit less shedding of fur as compared to the same animal prior to supplementation (for example, as described in canines in Example 1 and seen in FIGS. 7A and 7B). Moreover, the animal's coat color and observed coat shine may improve as compared to the same animal prior to supplementation (as described in canines in Example 1 and seen in FIGS. 9A and 9B).

Bioavailability of trace minerals depends on the physical and/or chemical properties of the form in which the metal is present in the diet. Bioavailability may be increased by associating the trace mineral with an organic molecule known as a ligand. The increased bioavailability of the trace mineral is generally the result of increased solubility, greater stability in the gut, enhanced absorption into circulation, and/or improved metabolic utilization.

### Mixed Amino Acid Complexes

Amino Acids are also effective ligands for nutritional delivery of trace minerals to animals. A mixed amino acid ligand of lysine and glutamic acid is particularly desirable for this purpose because of its high solubility and cost efficiency. The mixed amino acid ligand has the following general formula:

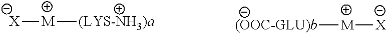

wherein M is the trace mineral ion (zinc, iron, manganese, copper), a and b are numbers selected so the ratio of LYS:GLU is within the range of from about 2:1 to about 1:2, preferably from about 1.5:1 to about 1:1:5, most preferably about 1:1, and X is an anion selected to balance the metal ion charge. Further information regarding amino acid complexes and methods of making same are provided in U.S. Pat. No. 8,741,375, which is incorporated herein by reference for this purpose.

For the purposes of the present disclosure, “mixed amino acid complex” means a trace mineral ligand complex of lysine and glutamic acid. The ratio of lysine to glutamic acid may be within the range of from about 2:1 to about 1:2, preferably from about 1.5:1 to about 1:1:5, most preferably about 1:1 of lysine to glutamic acid.

Accordingly, provided herein are trace mineral supplements and methods related to same. In some embodiments of the present disclosure, the mineral supplement comprises a copper mixed amino acid complex, an iron mixed amino acid complex, a manganese mixed amino acid complex, and a zinc mixed amino acid complex.

The mineral supplement may include at least two, at least three, or all four of a copper mixed amino acid complex, an iron mixed amino acid complex, a manganese mixed amino acid complex, and a zinc mixed amino acid complex.

In some embodiments, the zinc complex is in an amount between about 6 wt. % and about 10 wt. % of the mineral supplement, between about 7 wt. % and about 9.5 wt. % of the mineral supplement, or between about 7.5 wt. % and about 9 wt. % of the mineral supplement.

In some embodiments, the iron complex is in an amount between about 2.5 wt. % and about 5 wt. % of the mineral supplement, between about 3 wt. % and about 4.5 wt. % of the mineral supplement, or between about 3 wt. % and about 4 wt. % of the mineral supplement.

In some embodiments, the manganese complex is in a concentration of between about 1 wt. % and about 4 wt. % of the mineral supplement, between about 1.5 wt. % and about 3 wt. % of the mineral supplement, or between about 1.5 wt. % and about 2.5 wt. % of the mineral supplement.

In some embodiments, the copper complex is in a concentration of between about 0.2 wt. % and about 2 wt. % of the mineral supplement, between about 0.2 wt. % and about 1.5 wt. % of the mineral supplement, or between about 0.3 wt. % and about 1 wt. %. of the mineral supplement.

In some embodiments, the mineral complexes are included in the concentrations presented in Table 1. The mineral supplement may include at least two, at least three, or all four of the mixed amino acid mineral complexes in the ranges recited in Table 1.

TABLE 1

| Mixed Amino Acid | | More | Most |
|---|---|---|---|
| Mineral Complex | Preferred | Preferred | Preferred |
| Zinc | 6-10 | 7-9.5 | 7.5-9 |
| Iron | 2.5-5 | 3-4.5 | 3-4 |
| Manganese | 1-4 | 1.5-3 | 1.5-2.5 |
| Copper | 0.2-2 | 0.2-1.5 | 0.3-1 |

All values presented in wt. %

In some embodiments, the mineral supplement comprises the zinc, iron, manganese and copper complexes in a ratio between about 8:3:2:0.5 to about 10:4:3:1.

In some embodiments, the mineral supplement comprises a mixture of mixed amino acid mineral complexes and trace minerals in their inorganic form. The mineral supplement may comprise at least one, at least two, or at least three of a mixed amino acid complex and at least one, at least two, or at least three of a trace mineral in its inorganic form. One having skill in the art would appreciate that any combination of mixed amino acid mineral complexes and inorganic trace minerals could be used.

In some embodiments, the diet of a canine or feline is nutritionally supplemented by feeding the canine or feline the mineral supplement. In some embodiments, the feeding comprises providing a pet food comprising the mineral supplement. In some embodiments, the mineral supplement is added to the pet food at a weight ratio of between about 1:200 and about 1:1300, between about 1:300 and about 1:1000, between about 1:400 and about 1:900, or between about 1:450 and about 1:850 of the mineral supplement to the pet food. The mineral supplement may be added during the pet food manufacturing process. Alternatively, the mineral supplement may be added after the manufacturing process, for example after processing at the manufacturing plant or at home by the consumer prior to feeding to the canine or feline. Pet food according to the present disclosure is discussed in more detail in the following section.

In an embodiment, the mineral supplement is fed to a canine or feline to reduce shedding in the canine or feline as compared to the same animal prior to supplementation. In some embodiments, the mineral supplement improves the canine or feline's coat color and shine as compared to the same animal prior to supplementation.

### Pet Food and Methods of Manufacture

Provided herein are methods of manufacturing a pet food for a canine or a feline. In some embodiments, the method comprises adding a mineral supplement of the present disclosure to the pet food, wherein the mineral supplement comprises a copper mixed amino acid complex, an iron mixed amino acid complex, a manganese mixed amino acid complex, and a zinc mixed amino acid complex.

The pet food may include at least one, at least two, at least three, or all four of a copper mixed amino acid complex, an iron mixed amino acid complex, a manganese mixed amino acid complex, and a zinc mixed amino acid complex.

In some embodiments, the pet food comprises one or more of chicken, lamb, beef, pork, salmon, corn, a grain, a probiotic, a starch, a vegetable, a fruit, an animal fat, a seed oil, and a vegetable oil.

Pet food of the present disclosure may contain meat, meat by-products, seafood, dairy, eggs, etc. Meats may include, for example, the flesh of poultry, fish, and mammals (e.g., cattle, pigs, sheep, goats, and the like). Meat by-products include, for example, lungs, kidneys, brain, livers, and stomachs and intestines (freed of all or essentially all their contents). Protein may be intact, almost completely hydrolyzed, or partially hydrolyzed.

In some embodiments, the pet food comprises grain. The grain may be wheat, corn, barley, rice, sorghum, peanuts, oats, millet, wheat germ, corn germ, soybeans, cottonseed, oils derived therefrom, or a combination thereof.

In certain embodiments, the pet food comprises a binding agent. Suitable binding agents include, but are not limited to sodium alginate, gum arabic, sodium carboxymethyl cellulose, guar gum, xanthan gum, maltodextrin, pregelatinized starch, and/or soy protein binder. The starch source may be substantially non-gelatinized.

In some embodiments, the pet food may comprise one or more of oat fiber, cellulose, peanut hulls, beet pulp, parboiled rice, corn starch, corn gluten meal, and any combination of these.

In certain embodiments, the pet food may comprise one or more of chicory root, psyllium, pectin, blueberry, cranberry, squash, apples, oats, beans, citrus, barley, peas, celery, green beans, cauliflower, potato skins, fruit skins, vegetable skins, peanut hulls, and soy fiber.

Pet food of the present disclosure may also include vitamins and minerals in amounts required to avoid deficiency and maintain health. These amounts and methods of measurement are known by those skilled in the art. For example, AAFCO provides recommended amounts of such ingredients for dogs and cats. As contemplated herein, useful vitamins may include, but are not limited to, vitamin A, vitamin B1, B2, B3, B5, B6, B7, B9 and B12, vitamin C, vitamin D, vitamin E, vitamin H (biotin), vitamin K, folic acid, inositol, niacin, and pantothenic acid.

In certain embodiments, the pet food may comprise additional ingredients such as fillers, palatability enhancers, binding agents, flavors, stabilizers, emulsifiers, sweeteners, colorants, buffers, salts, coatings, and the like known to skilled artisans. Stabilizers include substances that tend to increase the shelf life of the composition such as preservatives, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Specific amounts for each composition component, food ingredient, and other ingredients will depend on a variety of factors such as the particular components and ingredients included in the composition; the species of animal; the animal's age, body weight, general health, sex, and diet; the animal's consumption rate; the type of disease or condition being treated, if any; and the like. Therefore, the component and ingredient amounts may vary widely and may deviate from the embodiments described herein.

In some embodiments, the mineral supplement may be added to the pet food. In some embodiments, the mineral supplement may be added to the pet food by a compounder or manufacturer at a site or by an animal's caregiver prior to feeding the animal. In some embodiments, the mineral supplement may be added during the processing of the pet food, such as during and/or after mixing of other components of the composition that is then packaged and made available to consumers. Such processing may include extrusion, canning, baking, and the like or any other method or process of producing pet foods that is known in the art.

The pet food may be prepared in a dry form using conventional processes known to skilled artisans. Typically, dry ingredients such as animal protein, plant protein, grains, and the like are ground and mixed together. Moist or liquid ingredients, including fats, oils, animal protein, water, and the like are then added to and mixed with the dry mix. The mixture is then processed into kibbles or similar dry pieces. Kibble is often formed using an extrusion process in which the mixture of dry and wet ingredients is subjected to mechanical work at a high pressure and temperature and forced through small openings and cut off into kibble by a rotating knife. The wet kibble is then dried and optionally coated with one or more topical coatings such as flavors, fats, oils, powders, and the like. Kibble also can be made from the dough using a baking process, rather than extrusion, wherein the dough is placed into a mold before dry-heat processing.

The pet food may be prepared in a canned or wet form using conventional food preparation processes known to skilled artisans. Typically, ground animal proteinaceous tissues are mixed with the other ingredients such as fish oils, cereal grains, balancing ingredients, special purpose additives (e.g., vitamin and mineral mixtures, inorganic salts, cellulose and beet pulp, bulking agents, and the like) and water in amounts Sufficient for processing. These ingredients are mixed in a vessel suitable for heating while blending the components. Heating of the mixture is affected using any suitable manner, for example, direct steam injection or using a vessel fitted with a heat exchanger. When heated to the appropriate temperature, the material will typically be in the form of a thick liquid. The thick liquid is filled into cans. A lid is applied, and the container is hermetically sealed. The sealed can is then placed into conventional equipment designed to sterilize the contents.

Pet foods of any consistency or moisture content are contemplated, e.g., the compositions of the present disclosure may be a moist, semi-moist, or dry animal food composition. “Moist” food refers to food that has a moisture content of 60 to 90% or greater. “Dry” food refers to compositions with 3 to 11% moisture content and is often manufactured in the form of small bits or kibbles. “Semi-moist” refers to compositions with 25-35% moisture content. Also contemplated herein are pet foods that may comprise components of various consistency as well as components that may include more than one consistency, for example, soft, chewy meat-like particles as well as kibble having an outer cereal component and an inner cream component as described in, e.g., U.S. Pat. No. 6,517,877.

As described above, in the case of equine pets, pet food can include kibble, but can also include, distillers' fermentation solubles, feed grains, corn cob flour, hay, rice, flaxseed, soybeans, soybean stalks, whey, and other cellulosic feed materials.

### Example Embodiments

The inventions are defined in the claims. However, below is a non-exhaustive list of non-limiting embodiments in numbered format. Any one or more of the features of these embodiments may be combined with any one or more features of another example, embodiment, or aspect described herein. Accordingly, the following numbered embodiments form part of the present disclosure but do not form part of the claims:

1. A method of nutritionally supplementing the diet of a canine, equine or feline comprising: feeding the canine, equine, or feline a mineral supplement comprising a copper mixed amino acid complex, an iron mixed amino acid complex, a manganese mixed amino acid complex, and a zinc mixed amino acid complex; wherein the zinc complex is in an amount between about 6 wt. % and about 10 wt. %; wherein the iron complex is in an amount between about 2.5 wt. % and about 5 wt. %; wherein the manganese complex is in a concentration of between about 1 wt. % and about 4 wt. %; and wherein the copper complex is in a concentration of between about 0.2 wt. % and about 2 wt. %.

2. The method of embodiment 1, wherein the zinc complex is in an amount between about 7.5 wt. % and about 9 wt. %; wherein the iron complex is in an amount between about 3 wt. % and about 4 wt. %; wherein the manganese complex is in a concentration of between about 1.5 wt. % and about 2.5 wt. %; and wherein the copper complex is in a concentration of between about 0.3 wt. % and about 1 wt. %.

3. The method of embodiment 1 or 2, wherein the mineral supplement comprises the zinc, iron, manganese and copper complexes in a ratio between about 8:3:2:0.5 to about 10:4:3:1.

4. The method of any one of embodiments 1-3, wherein the feeding comprises providing a pet food comprising the mineral supplement.

5. The method of embodiment 4, wherein the ratio of the mineral supplement to the pet food on a weight basis is between about 1:200 and about 1:1300.

6. The method of embodiment 4 or 5, wherein the ratio of the mineral supplement to the pet food on a weight basis is between about 1:450 and about 1:850.

7. A method of reducing shedding in a canine, equine or feline comprising: feeding the canine, equine or feline a mineral supplement comprising a copper mixed amino acid complex, an iron mixed amino acid complex, a manganese mixed amino acid complex, and a zinc mixed amino acid complex; wherein the zinc complex is in an amount between about 6 wt. % and about 10 wt. %; wherein the iron complex is in an amount between about 2.5 wt. % and about 5 wt. %; wherein the manganese complex is in a concentration of between about 1 wt. % and about 4 wt. %; and wherein the copper complex is in a concentration of between about 0.2 wt. % and about 2 wt. %.

8. The method of embodiment 7, wherein the zinc complex is in an amount between about 7.5 wt. % and about 9 wt. %; wherein the iron complex is in an amount between about 3 wt. % and about 4 wt. %; wherein the manganese complex is in a concentration of between about 1.5 wt. % and about 2.5 wt. %; and wherein the copper complex is in a concentration of between about 0.3 wt. % and about 1 wt. %.

9. The method of embodiment 7 or 8, wherein the mineral supplement comprises the zinc, iron, manganese and copper complexes in a ratio between about 8:3:2:0.5 to about 10:4:3:1.

10. The method of any one of embodiments 7-9, wherein the feeding comprises providing a pet food comprising the mineral supplement.

11. The method of embodiment 10, wherein the ratio of the mineral supplement to the pet food on a weight basis is between about 1:200 and about 1:1300.

12. The method of embodiment 10 or 11, wherein the ratio of the mineral supplement to the pet food on a weight basis is between about 1:450 and about 1:850.

13. The method of any one of embodiments 7-12, wherein the mineral supplement improves the canine or the feline coat color and shine.

14. A method of manufacturing a pet food for a canine, equine or a feline comprising: adding a mineral supplement to the pet food; wherein the mineral supplement comprises a copper mixed amino acid complex, an iron mixed amino acid complex, a manganese mixed amino acid complex, and a zinc mixed amino acid complex.

15. The method of embodiment 14, wherein the zinc complex is in an amount between about 6 wt. % and about 10 wt. % of the mineral supplement; wherein the iron complex is in an amount between about 2.5 wt. % and about 5 wt. % of the mineral supplement; wherein the manganese complex is in a concentration of between about 1 wt. % and about 4 wt. % of the mineral supplement; and wherein the copper complex is in a concentration of between about 0.2 wt. % and about 2 wt. % of the mineral supplement.

16. The method of embodiment 14 or 15, wherein the zinc complex is in an amount between about 7.5 wt. % and about 9 wt. %; wherein the iron complex is in an amount between about 3 wt. % and about 4 wt. %; wherein the manganese complex is in a concentration of between about 1.5 wt. % and about 2.5 wt. %; and wherein the copper complex is in a concentration of between about 0.3 wt. % and about 1 wt. %.

17. The method of any one of embodiments 14-16, wherein the mineral supplement comprises the zinc, iron, manganese and copper complexes in a ratio between about 8:3:2:0.5 to about 10:4:3:1.

18. The method of any one of embodiments 14-17, wherein the mineral supplement is added to the pet food at a weight ratio of between about 1:200 and about 1:1300 of the mineral supplement to the pet food.

19. The method of embodiment 18, wherein the mineral supplement is added to the pet food at a weight ratio of between about 1:450 and about 1:850 of the mineral supplement to the pet food.

20. The method of any one of embodiments 1-19, wherein the pet food comprises one or more of chicken, lamb, beef, pork, salmon, corn, a grain, a probiotic, a starch, a vegetable, a fruit, an animal fat, a seed oil, and a vegetable oil.

21. The method of embodiment 20, wherein the pet food is kibble.

22. A method of nutritionally supplementing the diet of a canine or feline comprising: feeding the canine or feline a mineral supplement comprising a copper amino acid complex, an iron amino acid complex, a manganese amino acid complex, and a zinc amino acid complex; wherein the zinc complex is in an amount between about 3 wt. % and about 8 wt. %; wherein the iron complex is in an amount between about 1 wt. % and about 4 wt. %; wherein the manganese complex is in a concentration of between about 0.5 wt. % and about 3 wt. %; and wherein the copper complex is in a concentration of between about 0.1 wt. % and about 2 wt. %.

23. The method of embodiment 22, wherein the zinc complex is in an amount between about 4.5 wt. % and about 6 wt. %; wherein the iron complex is in an amount between about 1.5 wt. % and about 3 wt. %; wherein the manganese complex is in a concentration of between about 1 wt. % and about 2 wt. %; and wherein the copper complex is in a concentration of between about 0.2 wt. % and about 1 wt. %.

24. The method of embodiment 22 or 23, wherein the mineral supplement comprises the zinc, iron, manganese and copper complexes in a ratio between about 5:2:1:0.3 to about 8:3:2:1.

25. The method of any one of embodiments claim 22-24, wherein the feeding comprises providing a pet food comprising the mineral supplement.

26. The method of embodiment 25, wherein the ratio of the mineral supplement to the pet food on a weight basis is between about 1:200 and about 1:1300.

27. The method of embodiment 25 or 26, wherein the ratio of the mineral supplement to the pet food on a weight basis is between about 1:450 and about 1:850.

28. A method of reducing shedding in a canine or feline comprising: feeding the canine or feline a mineral supplement comprising a copper amino acid complex, an iron amino acid complex, a manganese amino acid complex, and a zinc amino acid complex; wherein the zinc complex is in an amount between about 3 wt. % and about 8 wt. %; wherein the iron complex is in an amount between about 1 wt. % and about 4 wt. %; wherein the manganese complex is in a concentration of between about 0.5 wt. % and about 3 wt. %; and wherein the copper complex is in a concentration of between about 0.1 wt. % and about 2 wt. %.

29. The method of embodiment 28, wherein the zinc complex is in an amount between about 4.5 wt. % and about 6 wt. %; wherein the iron complex is in an amount between about 1.5 wt. % and about 3 wt. %; wherein the manganese complex is in a concentration of between about 1 wt. % and about 2 wt. %; and wherein the copper complex is in a concentration of between about 0.2 wt. % and about 1 wt. %.

30. The method of embodiment 28 or 29, wherein the mineral supplement comprises the zinc, iron, manganese and copper complexes in a ratio between about 5:2:1:0.3 to about 8:3:2:1.

31. The method of any one of embodiments 28-30, wherein the feeding comprises providing a pet food comprising the mineral supplement.

32. The method of embodiment 31, wherein the ratio of the mineral supplement to the pet food on a weight basis is between about 1:200 and about 1:1300.

33. The method of embodiment 31 or 32, wherein the ratio of the mineral supplement to the pet food on a weight basis is between about 1:450 and about 1:850.

34. The method of any one embodiments 28-33, wherein the mineral supplement improves the canine or the feline coat color and shine.

35. A method of manufacturing a pet food for a canine or a feline comprising: adding a mineral supplement to the pet food; wherein the mineral supplement comprises a copper amino acid complex, an iron amino acid complex, a manganese amino acid complex, and a zinc amino acid complex.

36. The method of embodiment 35, wherein the zinc complex is in an amount between about 3 wt. % and about 8 wt. % of the mineral supplement; wherein the iron complex is in an amount between about 1 wt. % and about 4 wt. % of the mineral supplement; wherein the manganese complex is in a concentration of between about 0.5 wt. % and about 3 wt. % of the mineral supplement; and wherein the copper complex is in a concentration of between about 0.1 wt. % and about 2 wt. % of the mineral supplement.

37. The method of embodiment 35 or 36, wherein the zinc complex is in an amount between about 4.5 wt. % and about 6 wt. %; wherein the iron complex is in an amount between about 1.5 wt. % and about 3 wt. %; wherein the manganese complex is in a concentration of between about 1 wt. % and about 2 wt. %; and wherein the copper complex is in a concentration of between about 0.2 wt. % and about 1 wt. %.

38. The method of any one of embodiments 35-37, wherein the mineral supplement comprises the zinc, iron, manganese and copper complexes in a ratio between about 5:2:1:0.3 to about 8:3:2:1.

39. The method of any one of claims 35-38, wherein the mineral supplement is added to the pet food at a weight ratio of between about 1:200 and about 1:1300 of the mineral supplement to the pet food.

40. The method of embodiment 39, wherein the mineral supplement is added to the pet food at a weight ratio of between about 1:450 and about 1:850 of the mineral supplement to the pet food.

41. The method of any one of embodiments 22-40, wherein the pet food comprises one or more of chicken, lamb, beef, pork, salmon, corn, a grain, a probiotic, a starch, a vegetable, a fruit, an animal fat, a seed oil, and a vegetable oil.

42. The method of embodiment 41, wherein the pet food is kibble.

43. A method of improving the mane of an equine comprising: feeding the equine a mineral supplement comprising a copper mixed amino acid complex, an iron mixed amino acid complex, a manganese mixed amino acid complex, and a zinc mixed amino acid complex; wherein the zinc complex is in an amount between about 6 wt. % and about 10 wt. %; wherein the iron complex is in an amount between about 2.5 wt. % and about 5 wt. %; wherein the manganese complex is in a concentration of between about 1 wt. % and about 4 wt. %; and wherein the copper complex is in a concentration of between about 0.2 wt. % and about 2 wt. %.

44. The method of embodiment 43, wherein the zinc complex is in an amount between about 7.5 wt. % and about 9 wt. %; wherein the iron complex is in an amount between about 3 wt. % and about 4 wt. %; wherein the manganese complex is in a concentration of between about 1.5 wt. % and about 2.5 wt. %; and wherein the copper complex is in a concentration of between about 0.3 wt. % and about 1 wt. %.

45. The method of embodiment 43 or 44, wherein the mineral supplement comprises the zinc, iron, manganese and copper complexes in a ratio between about 8:3:2:0.5 to about 10:4:3:1.

46. The method of any one of embodiments 43-45, wherein the feeding comprises providing a pet food comprising the mineral supplement.

47. The method of embodiment 46, wherein the ratio of the mineral supplement to the pet food on a weight basis is between about 1:200 and about 1:1300.

48. The method of embodiment 46 or 47, wherein the ratio of the mineral supplement to the pet food on a weight basis is between about 1:450 and about 1:850.

49. The method of any one of embodiments 43-48, wherein the mineral supplement improves the mane length of the equine.

50. The method of any one of embodiments 43-49, wherein the pet food comprises hay.

51. The method of any one of embodiments 1-50, wherein the mixed amino acid complex comprises lysine and glutamic acid in a ratio of from about 2:1 to about 1:2.

52. The method of embodiment 51, wherein the ratio of lysine to glutamic acid is about 1:1.

### EXAMPLES

Embodiments of the nutritional supplements and methods of using the same are further defined in the following non-limiting Examples. It should be understood that these Examples, while indicating one or more preferred embodiments, are given by way of illustration only and are non-limiting. From the above discussion and these Examples, one skilled in the art can ascertain the essential characteristics of the invention(s), and without departing from the spirit and scope thereof, can make various changes and modifications of the embodiments of the invention to adapt it to various usages and conditions. Thus, various modifications of the embodiments, in addition to those shown and described herein, will be apparent to those skilled in the art from the foregoing description. Such modifications are also intended to fall within the scope of the appended claims.

### Example 1

A 90-day feeding trial was conducted to investigate isolevel effects of different trace mineral sources on various health parameters in 28 large breed (Golden Retriever, Labrador Retriever, and Rottweiler) senior dogs aged 5.81 to 14.01 years. The experiment was a Randomized Complete Block Design. A total of 28 dogs (10 male, 18 female) were in the study, including 4 Rottweiler, 6 Golden Retriever, and 18 Labrador Retriever breeds. Average age of the dogs in the study was 9.8 years±4 years at the beginning of the study.

Dogs were blocked by breed, sex, and age then randomly assigned to either a Control group or treatment group, as presented in Table 2.

TABLE 2
Treatment Groups

| | No. Dogs/ | Supplemental Dietary |
|---|---|---|
| Treatment | Treatment | Mineral, mg/kg |
| Control | 11 total | 100 ppm Zn |
| | R: 2 | 7.2 ppm Mn |
| | GR: 3 | 7.3 ppm Cu |
| | LR: 6 | 88 ppm Fe |
| | M: 4, F: 7 | |
| Treatment 1 | 17 total | 100 ppm Zn |
| | R: 2 | 7.2 ppm Mn |
| | GR: 3 | 7.3 ppm Cu |
| | LR: 12 | 88 ppm Fe |
| | M: 6, F: 11 | |
| Total | 28 | |

R = Rottweiler,
GR = Golden Retriever,
LR = Labrador Retriever,
M = Male,
F = Female

Dogs had a 14-day wash-out period, during which all dogs consumed the Control diet prior to the start of the trial. Employees handling the dogs had no knowledge of specific treatment characteristics. Test diets were commercially formulated canine diets that meet or exceed AAFCO Dog Food Nutrient Profiles for Adult Maintenance (AAFCO OP 2020). The only variable between treatments was the trace mineral premix and trace mineral source.

Trace mineral concentrations in each treatment diet are presented in Table 3. Trace minerals in the Control group were delivered via traditional sulfate complexes. Trace minerals in Treatment 1 were delivered via mixed amino-acid complexes. Trace mineral premix ingredient content for each group was as follows:

Control: CaCO3, ZnSO4 (Zn, 8.8%), FeSO4 (Fe, 3.9%), CuSO4 (Cu, 1.1%), MnO (Mn, 5684 ppm), Ca Iodate (I, 1584 ppm), NaSeO3 (Se, 310 ppm)

Treatment 1: Commercially available mineral complexes were obtained and used. Specifically, a zinc mixed amino acid complex (Zn, 8.44%), an iron mixed amino acid complex (Fe, 3.57%), a manganese mixed amino acid complex (Mn, 2.13%), and a copper mixed amino acid complex (Cu, 0.59%) were obtained and combined along with Ca Iodate (I, 905 ppm) and NaSeO3 (Se, 318 ppm). The resultant mixture was added to a pet food and the concentration of minerals in the food product is provided in Table 3.

Vitamin premix ingredient content was as follows for all treatments: Vitamin A, 17,163,000 IU/kg; Vitamin D3, 920,000 IU/kg; Vitamin E, 79,887 IU/kg; Vitamin B12, 22 mg/kg; Riboflavin, 4,719 mg/kg; Pantothenic acid, 12,186 mg/kg; Niacin, 64,736 mg/kg; Folic acid, 720 mg/kg; Pyridoxine, 5,537 mg/kg; Thiamine, 14,252 mg/kg; Biotin, 70 mg/kg.

TABLE 3
Trace Mineral Concentrations in Treatment Diets (Dry Matter)

| Mineral, ppm | Control | Treatment 1 |
|---|---|---|
| Zinc (Zn) | 159 | 158 |
| Iron (Fe) | 220 | 186 |
| Manganese (Mn) | 32 | 45 |
| Copper (Cu) | 19 | 16 |
| Iodine (I)* | 1.8 | 1.1 |
| Selenium (Se)* | 0.4 | 0.4 |

*Calculated

Diets were extruded under typical commercial conditions to ensure uniformity of kibbles. The test diets were mixed according to standard manufacturing practices. Pet food kibbles were manufactured under standard pet food extrusion conditions. Extrusion and drying conditions were not reported. Experimental diet ingredient mixes are presented in Table 4. Basal diet nutrient concentrations are presented in Table 5.

TABLE 4
Experimental Diet Ingredient Mixes

Treatment Inclusion, lb/ton

| Ingredient | Control | Treatment 1 |
|---|---|---|
| Chicken meal, reg. | 628.00 | 628.00 |
| ash 15% | | |
| Milo whole US#2 | 451.00 | 451.00 |
| Corn whole US#2 | 450.80 | 450.80 |
| Wheat US#2 | 437.30 | 437.30 |
| Salt mixing | 12.60 | 12.60 |
| Beet Pulp | 12.60 | 12.60 |
| KS D/C Vit 2016 | 4.00 | 4.00 |
| KS D/C TM NPRC | 2.40 | |
| 2016 | | |
| Treatment 1 Trace | | 2.50 |
| Mineral Pre-Mix | | |
| Choline Chloride | 1.00 | 1.00 |
| 60% | | |
| Oxy-gon Powder | 0.30 | 0.30 |

TABLE 5
Basal Diet Nutrient Concentrations (As Fed)*

| Item | % |
|---|---|
| Moisture | 9.76 |
| Crude Protein | 28.40 |
| Crude Fat | 15.10 |
| Crude Fiber | 1.50 |
| Ash | 7.14 |
| Calcium | 1.50 |
| Phosphorus | 1.00 |

*Additional fat was added topically post-extrusion and is included in this analysis

### Feeding Regime

Days 1 to 14: 2-week wash-out/acclimation period for all dogs on study. All dogs received the Control diet containing sulfate/oxide trace minerals to allow for diet adaptation and any physiological disruption due to change of the food.

Days 15 to 105: Study period. Dogs were offered about 650 g/day of the assigned treatment diets (Control or Treatment 1) for the remainder of the study, from day 15 to day 105. Dogs were offered sufficient amount of food to maintain their body weight, and food intake was recorded daily. Dogs were allowed ad libitum access to drinking water. Dogs were fed once daily.

### Measured Parameters

The following parameters were observed and/or measured during the course of the study according to the methods presented and described below.

### Performance

Daily food consumption, general health observations, and abnormalities were recorded. Exercise/socialization reports were recorded three times daily (morning, mid-day, afternoon). Individual body weight and body condition scores (BCS) were measured and recorded on a weekly basis. Each dog's activity was monitored with a FitBark™ accelerometer attached to the dog's collar. FitBark™ monitors were applied to dogs one month prior to the onset of treatment diets.

### Skin

Transepidermal water loss (TEWL), skin elasticity and retraction time, ultrasound of the abdominal skin, and Paw Pad and Heel were measured with DermaLab according to the User Manual of DermaLab at the beginning and the end of the study

### Hair

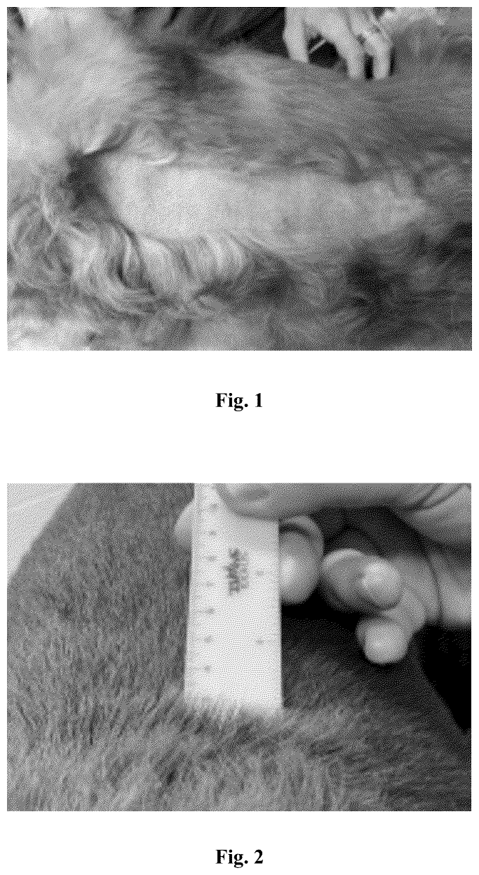

Point of evaluation was an area lateral to the spine, directly behind the shoulder for determination of hair length and growth. See FIG. 1 for an example of the hair growth point of evaluation. Initial hair length was measured from base of hair shaft at pre-shaved site (7.6 cm×15.2 cm area) in 3 different regions: about ⅓ front, mid-point, and about ⅓ back See FIG. 2 for an example of hair length evaluation. Growth rate was determined by monthly measurements of the aforementioned area.

A 7.6 cm wide×15.2 cm long area of coat was shaved with a 1.5 mm #10 blade as shown in FIG. 1. Hair samples were collected and placed in sample bags with individual dog ID information and date collected. Pictures were taken of the shaved area and other parts of the dog body for visual evaluation at the beginning and the end of the study. Hair samples collected at the beginning and end of study were evaluated for color with a WF30 handheld colorimeter and tensile measurements were taken, including cross-sectional area, mean diameter, Young's Modulus, break stress, and break extension.

### Shedding

Ten strokes with a grooming brush were performed at a designated area at the top of the back/lumbar region using a guide template about 30 cm in length. Shed hair samples were collected, weighed, photographed, and put in a sample bag with the dog's ID information and date collected and retained for future analysis.

For the duration of the study, weekly facility grooming protocol of grooming, bathing, and ear cleaning was discontinued and conducted only monthly after initial sample collections were taken.

### Sampling

Weather conditions were recorded on sampling days. The following sampling schedule was followed:
Day 15—Beginning of Test Period: Body Condition Score (BCS), individual body weight, shedding/grooming data, initial hair length measured, hair samples collected, photo of shaved area, DermaLab evaluation of skin, fecal samples.
Day 45: BCS, individual body weight, shedding/grooming data, hair measurements for growth rate.
Day 75: BCS, individual body weight, shedding/grooming data, hair measurements for growth rate.
Day 105—End of Test Period: BCS, individual body weight, final hair length measured, shedding/grooming data, hair samples collected, photo of shaved area, activity monitor removed, DermaLab evaluation of skin, fecal samples.

Photographic documentation of each dog was taken. Photographs of the front, top, sides, face, rear, and each paw was collected for each dog at the beginning and end of the test period.

### Results/Discussion

Overall, the study ran smoothly with dogs accepting their respective treatment diet and allowing employees to collect key metrics.

### Hair Growth

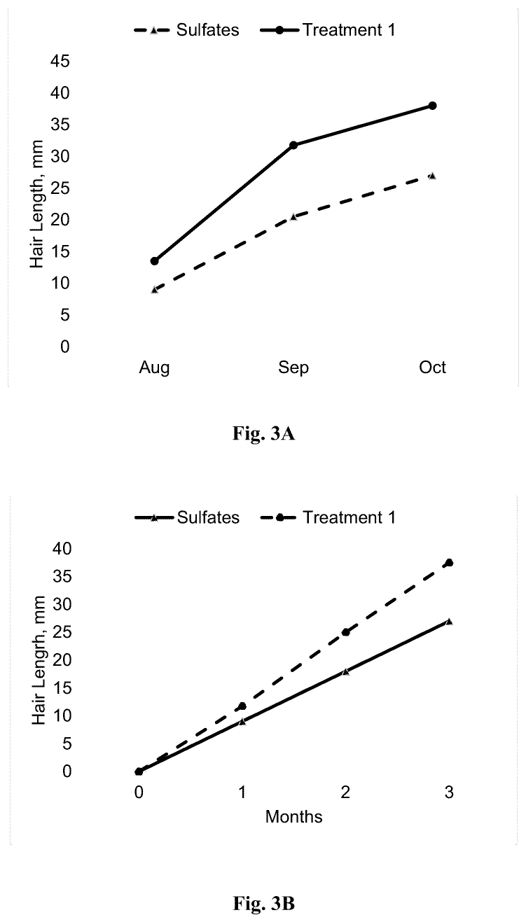

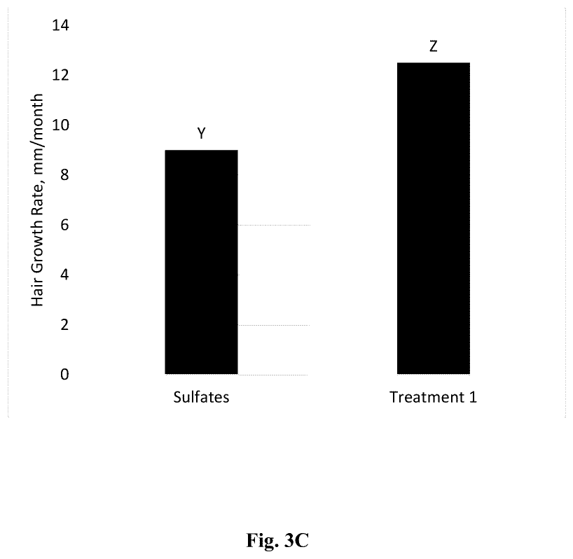

Hair growth in length and rate was significantly greater in dogs receiving diets fortified with Treatment 1 (mixed amino acid complexes) versus the sulfate Control (P<0.05). The results are represented graphically in FIGS. 3A, 3B, and 3C. Hair length showed a stronger response (28% greater length) than growth rate (23% faster) in mm/month for dogs receiving Treatment 1 vs. the Control (P<0.01).

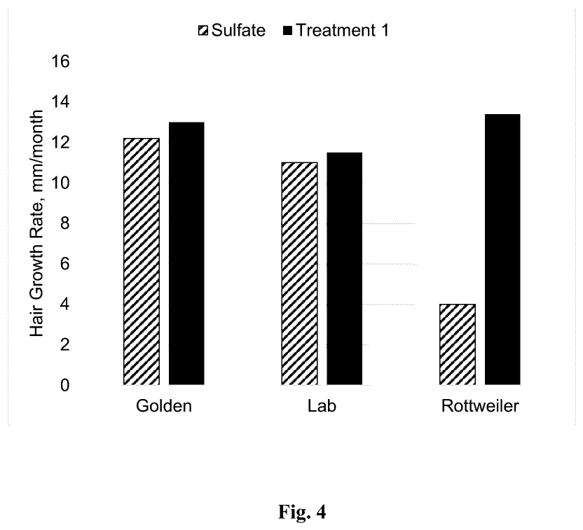

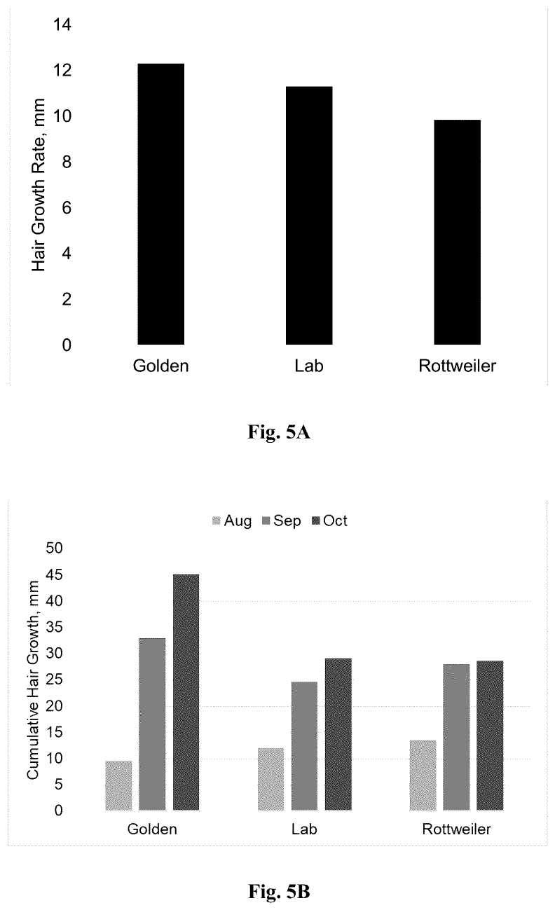

Though there were limited number of dogs in each treatment group, Rottweilers showed the greatest response in hair growth rate by treatment, responding more favorably to Treatment 1 (see FIG. 4). Yet overall, there were no significant breed, sex or interaction effects for hair growth rate (P>0.42). Not surprisingly, breeds differed in cumulative hair length effect with Golden Retrievers growing longer hair than Labs and Rottweilers (Breed P=0.02, Breed×Month P=0.001; see FIGS. 5A and 5B).

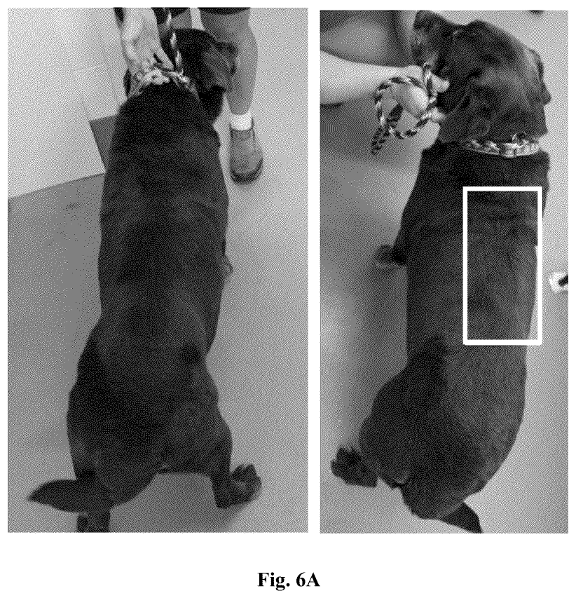

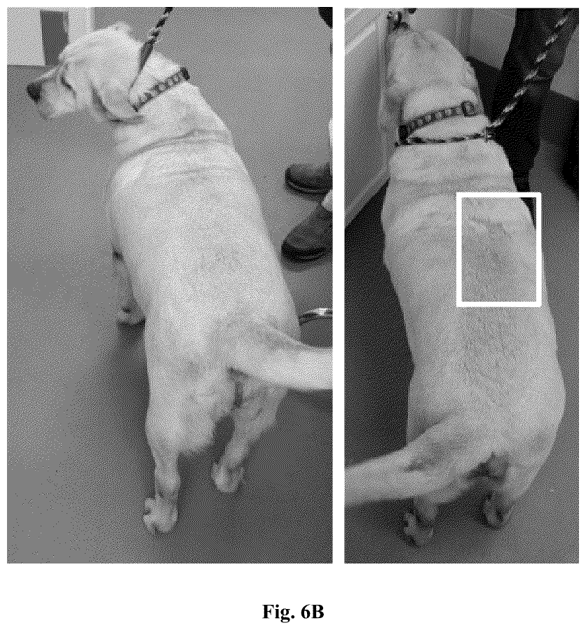

More dogs were observed with delayed or limited hair regrowth at the clipped location when receiving the Control treatment. FIGS. 6A and 6B depict dogs on the Control diet with delayed hair regrowth (Left, dog prior to shaving; Right, dog at end of study period). This response seemed to be more pronounced in Labrador Retrievers than Golden Retrievers or Rottweilers.

### Hair Shedding

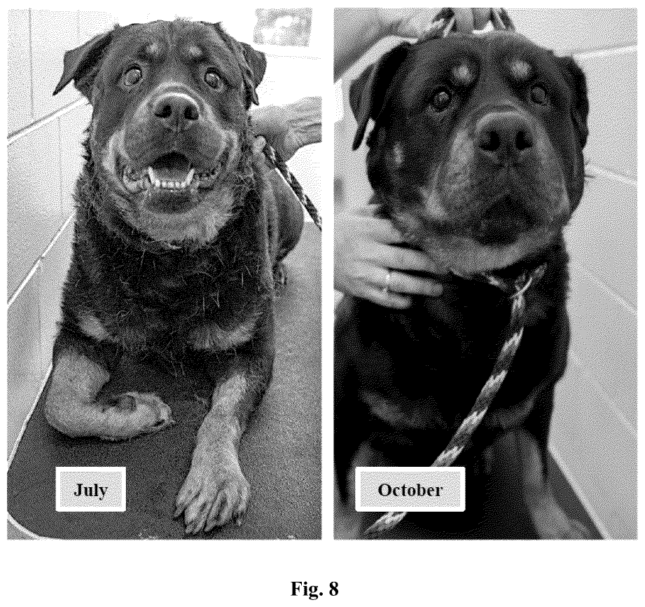

Shed hair results showed a reduction in hair loss for dogs receiving Treatment 1 (57% lower) compared to dogs receiving the Control treatment (see FIGS. 7A and 7B). Overall, there was a general trend of reduced hair shedding for dogs on both treatments while those receiving the Control declined similarly until September then increased in October (FIG. 7A). It does not appear weather was a major contributor to these responses as temperatures increased as the summer progressed with a peak in August (high in mid-90's, average 80-82 F) then cooled in September (high in mid to upper 80's, average 67 to 80 F). The shed hair graphs in FIGS. 7A and 7B show only three months of data as the July observation was used to covariately adjust the data. Monthly photos of the dogs helped verify changes in shedding as many dogs had significant shedding early in the study with later photos showing a visible decline in hair loss (see FIG. 8 depicting a dog before and after treatment with Treatment 1).

### Hair Color Improvement

Possibly the most remarkable response seen was a visible improvement in hair color and coat sheen in dogs receiving Treatment 1 vs. those supplemented with the Control. Dogs receiving Treatment 1 had darker (black and chocolate) or lighter white/blonde coloring, shinier hair and softer hair texture than dogs receiving the Control (see FIGS. 9A and 9B). FIG. 9A depicts a dog before and after treatment with Control. FIG. 9B depicts a dog before and after treatment with Treatment 1. Even though the kennel staff and researchers were blinded to the dietary treatments, the improvements in coat color and conditions of dogs receiving one of the two treatments were evident to all parties.

### Hair Color Measurements

Hair samples from each dog were evaluated for color and tensile measurements. The color of the hair samples was measured with a WF30 hand-held colorimeter after hair samples were washed and dried, which measures three-dimensional color space: L*—black to white, a*—green to red, and b*—blue to yellow. Delta E (ΔE=[(ΔL*)2+(Δa*)2+(Δb*)2]0.5) was compared among the Control and treatment group. No difference in delta E among the groups was found and the same was true for the changes in each dimension analyzed individually, ΔL*, Δa* and Δb*. However, Treatment 1 significantly reduced the delta E when compared with the Control in Golden Retriever and yellow-coated Labrador Retriever dogs, as presented below in Tables 6 and 7. These results indicate that Treatment 1 partially prevents the seasonal color change in Golden Retrievers and yellow-coated Labrador Retrievers that was observed in the Control group.

TABLE 6
Delta E in Golden Retrievers

| | Number of | Mean | Std. | Std. Err | | |
|---|---|---|---|---|---|---|
| Treatment | Dogs | (Delta E) | Deviation | Mean | Differences | |
| Control | 3 | 4.5 | 1.5 | 0.8 | A | |
| Treatment 1 | 3 | 2.4 | 0.5 | 0.3 | | B |

*Levels not connected by same letter are significantly different.

TABLE 7
Delta E in Yellow Labrador Retrievers

| | Number of | Mean | Std. | Std. Err | | |
|---|---|---|---|---|---|---|
| Treatment | Dogs | (Delta E) | Deviation | Mean | Differences | |
| Control | 3 | 4.4 | 1.3 | 0.8 | A | |
| Treatment 1 | 6 | 3.3 | 0.6 | 0.3 | | B |

*Levels not connected by same letter are significantly different.

In contrast to Golden Retrievers and yellow coated Labrador Retrievers, Treatment 1 increased delta E in black & chocolate color-coated Labrador Retriever dogs, which supports the visual observations of the hair coat color changes in those dogs. These results are presented in Table 8 below.

TABLE 8
Delta E in Dark-Coated (Black and Chocolate) Labrador Retrievers

| | Number of | Mean | Std. | Std. Err | | |
|---|---|---|---|---|---|---|
| Treatment | Dogs | (Delta E) | Deviation | Mean | Differences | |
| Control | 3 | 1.7 | 1.0 | 0.6 | | B |
| Treatment 1 | 6 | 3.1 | 1.2 | 0.5 | A | |

*Levels not connected by same letter are significantly different

Hair samples were also measured with a Dia-Stron Mini Tensile Tester (MTT) for various hair parameters, including cross-sectional area, mean diameter, Young's modulus, break stress, and break extension at controlled environmental conditions of 20±2° C. and 60±2% RH. Ten hair fibers longer than 40 mm from each dog sampling time points (July, pre-treatment and October, post-treatment) were measured with MTT. No statistically significant difference was observed in these measurements among the control and treatment group. However, hair cross-sectional area and mean diameter in Labrador Retriever dogs in the Treatment 1 group was relatively unchanged during the study while those two parameters were increased in Golden Retriever dogs in the Control group, as presented below in Tables 9 and 10. No difference was observed in other breeds of dogs used in this study.

TABLE 9
Cross-Sectional Area of Hairs in Golden Retrievers

| | Number of | Mean (% | Std. | Std. Err | | |
|---|---|---|---|---|---|---|
| Treatment | Dogs | difference) | Deviation | Mean | Differences | |
| Control | 3 | 36.0 | 20.6 | 11.9 | A | |
| Treatment 1 | 3 | −0.4 | 10.9 | 6.3 | | B |

*Levels not connected by same letter are significantly different.

TABLE 10
Mean Diameter of Hairs in Golden Retrievers

| | Number of | Mean (% | Std. | Std. Err | | |
|---|---|---|---|---|---|---|
| Treatment | Dogs | difference) | Deviation | Mean | Differences | |
| Control | 3 | 16.2 | 8.1 | 4.7 | A | |
| Treatment 1 | 3 | −0.4 | 5.1 | 2.9 | | B |

*Levels not connected by same letter are significantly different.

Skin Parameters Measured with DermaLab

In this study, skin integrity was evaluated using DermaLab probe techniques. Trans Epidermal Water Loss (TEWL) was determined via probe as the amount of water that passively evaporated through skin to the external environment due to water vapor pressure gradient on both sides of the skin barrier. This measure was used to characterize skin barrier functional integrity. Similarly, the following parameters were also measured with the DermaLab: skin elasticity, skin retraction time. No difference among dietary treatment groups was found in these skin parameter measurements.

### Paw Pads and the Abdominal Skin

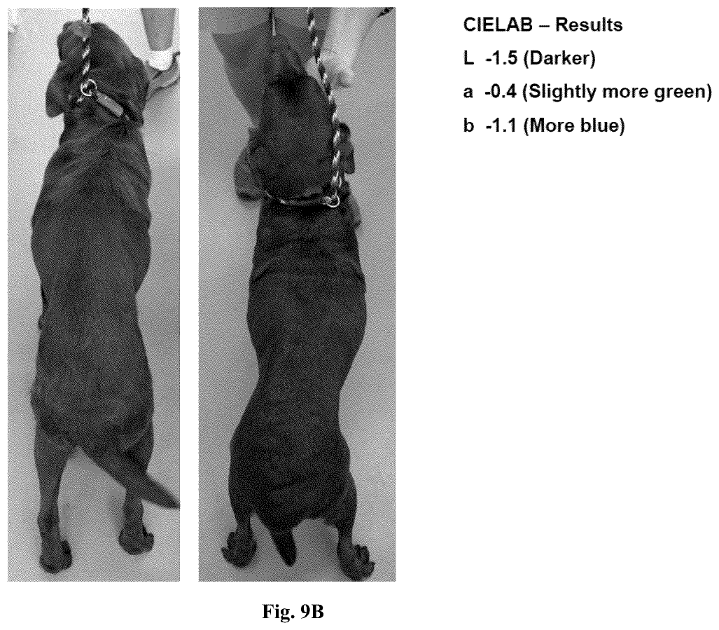

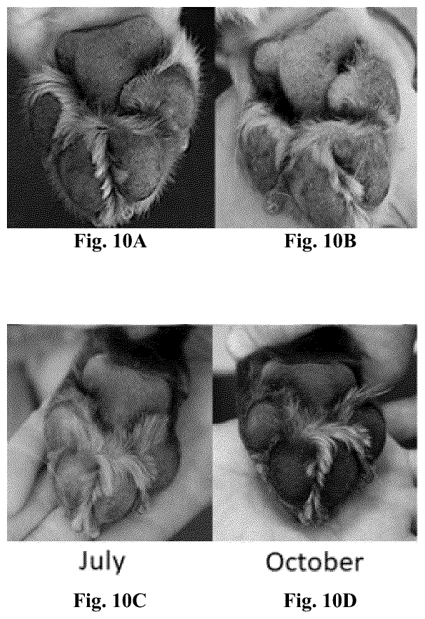

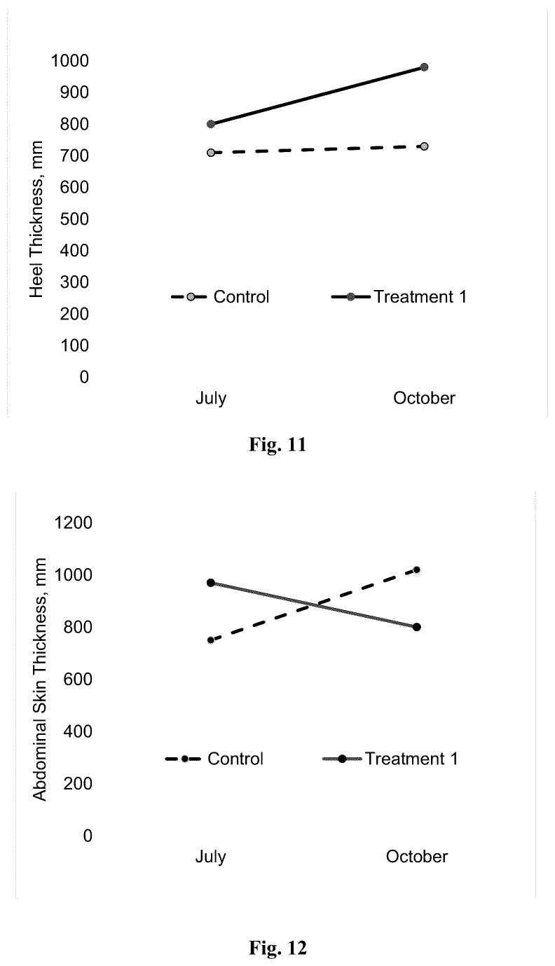

Visual evaluation showed a significant improvement in paw pad color and texture in dogs fed Treatment 1, suggesting healthier paw pads (see FIG. 10. Top, paw pad in dog before and after treatment with Control; Bottom, paw pad in dog before and after treatment with Treatment 1). Treatment 1 supplementation also significantly increased thickness of dog heel (treatment, P<0.01) (see FIG. 11) but decreased thickness of the lower abdominal skin (treatment*month, P=0.013; see FIG. 12), as measured with a DermaLab ultrasound. Since the abdominal skin was generally healthy during the study, the health implication of thinner abdominal skin induced by Treatment 1 supplementation is not fully understood.

### Example 2

Well-fed cats showing signs of trace mineral insufficiency is not common, yet zinc and other trace mineral absorption may be hindered by certain nutrients or food ingredients. Scientists have determined that high levels of calcium interfere with zinc assimilation. Zinc absorption is also inhibited by high levels of copper, cobalt, phytates (salts produced by phytic acid, and acid in cereal grains like corn, wheat, soybeans, peas, and legumes), phosphorous, starch, and fiber, all common components in commercial pet foods. Zinc insufficiency in cats is occasionally characterized by one or more of the following clinical signs: skin lesions, slow and sparse hair growth, conjunctivitis, emaciation, poor growth, abnormal sexual development, keratitis, digestive upset, and vomiting.

Previous works have demonstrated that supplementation of companion and livestock animals with organic trace minerals as metal amino acid complexes has resulted in improved trace mineral absorption and retention. Ultimately, these improvements in trace mineral bioefficacy have resulted in improvements in skin integrity, immune competence, hair length and density, and enhanced animal wellbeing.

Accordingly, a study was conducted to evaluate and compare the effects of trace mineral sources delivered in the forms of sulfate complexes (Control group) or mixed amino acid complexes on feline skin and coat health parameters in cats. Forty (40) cats total were involved in the study, with 20 in each treatment group.

Three dry, extruded, study diets were used and are presented below in Table 11. Diet 1 (Control) was offered to all cats during the acclimation period, whilst Diet 2 (Control) and Diet 3 (test) were offered during the test period. Diets 1 and 2 were identical but labeled differently for clarity between the pre-treatment and treatment phase. All diets were formulated to meet AAFCO Cat Food Nutrient Profiles for Adult Maintenance (2021). They were identical in ingredients and use levels except trace mineral premixes. The ingredients included corn, chicken meal, soybean meal, wheat middlings, wheat, corn gluten meal, poultry fat, natural flavors, fishmeal, brewer's yeast, dried whey, salt, mineral premix (test ingredients), choline chloride, DL-methionine, vitamin premix (vitamin E supplement, niacin supplement, thiamine mononitrate, calcium pantothenate, vitamin A supplement, pyridoxine hydrochloride, riboflavin supplement, vitamin D3 supplement, vitamin B12 supplement, folic acid, biotin) and calcium propionate.

TABLE 11
Dietary Treatments

| | | | Dietary |
|---|---|---|---|
| | Cats/ | | Supplement |
| Treatment | Group | Trace Mineral Source | mg/kg |
| Diet 1 | 40 | Zinc Sulfate Monohydrate, | 100 ppm Zn |
| (Pre- | | Manganese Sulfate Monohydrate, | 15 ppm Mn |
| feeding) | | Copper Sulfate Pentahydrate, Iron | 12 ppm Cu |
| | | Sulfate Dihydrate | 80 ppm Fe |
| Diet 2 | 20 | Zinc Sulfate Monohydrate, | 100 ppm Zn |
| | | Manganese Sulfate Monohydrate, | 15 ppm Mn |
| | | Copper Sulfate Pentahydrate, Iron | 12 ppm Cu |
| | | Sulfate Dihydrate | 80 ppm Fe |
| Diet 3 | 20 | Zinc Mixed Amino Acid Complex, | 100 ppm Zn |
| | | Manganese Mixed Amino Acid | 15 ppm Mn |
| | | Complex, Copper Mixed Amino | 12 ppm Cu |
| | | Acid Complex, Iron Mixed Amino | 80 ppm Fe |
| | | Acid Complex | |

### Experimental/Sampling Timeline

Days 1-14: Pre-feeding acclimation period; all 40 cats given Diet 1.
Days 15-105: Feeding/test period; 20 cats given Diet 2 and 20 cats given Diet 3.

Initial data collection on day 15 of study before switching diets. Middle data collection on day 60 of study. Final data collection on day 105 of study.

### Study Design

The study was a double blind, randomized complete block study design. Staff and researchers were blinded to dietary treatments. Forty apparently healthy adult cats, aged 5-14 inclusive, male and female, neutered or intact were used for the study. The duration of the study was 105 days. Cats were stratified into 2 groups of 20 cats each, based on sex, age, hair coat color, and body weight. All cats were fed Diet 1 for 14 days (day 1 to 14). Starting on day 15, one group was randomly assigned to receive Diet 2 and the other group was assigned to receive Diet 3 for an additional 90 days. Prior to study initiation, standard colony diet (Purina Cat Chow) was meal-fed, checked daily, and supplied in appropriate amounts according to body weight.

During the acclimation period, Diet 1 was offered once daily for a twenty-two-hour period at approximately 7:00 am each day and was the sole source of food for all felines. The initial amount of the study diet offered was based on dietary ME content and each individual animal's initial body weight (1.2×70(BWkg)0.75). Based on weekly body weights, food amounts were adjusted as needed to maintain a consistent body weight. The study diet was weighed in and out daily to determine food consumption. Exception to the 22-hour feed was on day 14 when the food was removed at the end of the day in preparation for the transition to Diets 2 or 3 on day 15.

During days 15-105, the amount of diet offered was adjusted weekly in order to maintain each animal's body weight. Any remaining diet was weighed at the end of the daily feeding period and food consumption was recorded. Food was made available for twenty-two (22) hours per day and fed at approximately the same time each day. Diets 2 and 3 were the sole sources of food provided to the cats throughout the test period of the study. Exception to the 22-hour feed was at the end of the day on days 59 and 104, prior to procedural/sample days. Food was not returned to the animals on procedural/sample days 60 and 105 until the procedures/samplings were completed on all cats on these days.

Food consumption was recorded daily, and body weights were recorded weekly. General health observations were recorded daily. Body condition scores (9-point scale), hair coat scores, shedding/grooming with shed hair collection, hair length measurements, body (front, back and side) hair coat and face (eyes and nose) pictures, chromameter measurements (L*, a*, b*), and paw pad/nail pictures were performed/taken on days 15, 60 and 105. Hair samples from a shaved area were collected on days 15 and 105.

In order to accommodate the sample and data collection activities required on days 15, 60, and 105, the start of the study was staggered over two days with 10 cats from each group (20 cats total per group) starting on each day. The staggered groups were balanced with dietary treatments, age, body weight, hair coat, and sex. To properly perform the activities required on days 15, 60, and 105, cats were anesthetized. Anesthesia allowed for the appropriate restraint required and reduced the chance of injury to technical personnel. Isoflurane (5%) in oxygen was used for induction. Cats were intubated and maintained on 2-3% isoflurane in oxygen with the exception of the eyes/nose picture that required the eyes to be open.

### Parameters

### Performance

Food consumption and general health was monitored and recorded daily. Clinical observations were performed twice daily to record anything unusual (i.e., abnormal stool quality, evidence of vomiting, abnormal behavior, poor coat quality, poor body condition, etc.). Cats were allowed to exercise and socialize as normal. Individual body weight was recorded weekly. Body condition score (9-point scale) was measured on days 0, 15, 60, and 105.

### Hair, Skin, and Paw Pads

An area lateral to the spine directly behind the shoulder blade running down the back (˜10 cm) was used as the point of evaluation to determine hair length and growth rate. Hair length was measured from base of hair shafts in a shaved 7.5 cm×10 cm area in 3 different regions: left ⅓, middle ⅓, and right ⅓ of the area. Measurements were taken on days 15, 60, and 105 of the study. The length of hair was measured from the base of the shaft to the tip of the hair at about a 45-degree angle. Three measurements at the left ⅓, middle ⅓, and right ⅓ of the shaved 7.5 cm×10 cm area were recorded and photographed. The average length of the longest guard hairs was also recorded.

High-definition photos of the shaved area for hair & skin assessments were taken immediately before and after clipping. Chromameter measurements (L*, a*, and b*) were conducted at four corners and the middle point of the shaved area before and after clipping at day 15, and clipped area at days 60 and 105.

Shedding measurements were taken at days 15, 60, and 105. Ten strokes were made with a grooming brush at a designated area at top of back/lumbar region from point of shoulder to base of tail about 20 cm in length. The brush/comb was weighed before shed hair collection (tare value) and after to determine rate of hair shedding. Shed hair samples were collected and retained for future analysis and stored at room temperature. All other normal grooming protocols were discontinued for the duration of the study.

Coat quality was measured on days 15, 60, and 105. High-definition photos were taken of individual cats from the top, side, and front (whole animal), face with focus on nose and eyes, separately. Photos were taken at uniform distance and light intensity to include a color reference card to standardize the photo to standard light value to eliminate any change in natural or room light. Hair coat was scored as follows: 0=Dull, coarse, dry; 1=Poorly reflective, non-soft; 2=Slightly reflective, somewhat soft; 3=Medium reflective, medium soft; 4=Highly reflective, very soft; 5=Greasy. Scale/dandruff was scored as follows: 0=None; 1=Fine over back; 2=Fine over body; 3=Medium over back; 4=Medium over body; 5=Severe. Overall haircoat was scored as follows: 1=Poor, 2=Satisfactory, 3=Good, 4=Excellent.

The paw pads were examined at days 15, 60, and 105. High-definition photos were taken of all 4 paws with focus on paw pads at uniform distance and light intensity with color reference card for standardization.

### Example 3

Testing was also performed to assess any benefit of the trace minerals when provided to horses. Horses were fed their standard rations which were supplemented by control trace minerals in sulfated form and trace minerals bound by the amino acid complexes disclosed herein. The dietary treatments are provided in Table 12.

TABLE 12
Equine Study Dietary Supplements

| | | Dietary |
|---|---|---|
| | | Supplement |
| | Trace Mineral Source | mg/kg |
| Control | Zinc Sulfate Monohydrate, | 425 ppm Zn |
| | Manganese Sulfate Monohydrate, | 425 ppm Mn |
| | Copper Sulfate Pentahydrate | 140 ppm Cu |
| Example | Zinc Mixed Amino Acid Complex, | 425 ppm Zn |
| Dietary | Manganese Mixed Amino Acid Complex, | 425 ppm Mn |
| Supplement | Copper Mixed Amino Acid Complex | 140 ppm Cu |

The horses had ad libitum access to pasture with a white salt block. The trace mineral supplement was added to 0.5-2.0 kg/hd/day of timothy hay. The supplements were provided a as a top-dress in powdered form.

### Measured Outcomes

We assess the hair growth for both the mane and body hair as well as hoof growth.

Mane hair length (cm): Mane clipped to based of hair follicle, 3″ up from withers. Hair length measured at days 0, 30, 60, and 90, with hair samples collected, and photos taken.

Shed hair volume: Collection area spanned 12 inches at the croup, where 10 strokes with a curry comb were performed on days 0, 30, 60, and 90. Hair samples were collected and weighed.

Hoof growth (cm): Hooves were notched ˜1 cm below the coronet band and measured with a micrometer on days 30, 60, and 90. Horses had farrier trims and shoe services at ˜35 days.

### Results

Data was analyzed using the Proc Glimmix procedure of SAS with individual horse as the experimental unit, day and treatment and their interaction as fixed effects, and day zero measurement for each parameter as the covariate (for all parameters except mane length and shed hair). The normality and homogeneity of the studentized residuals from the reported model were verified. Outliers were removed if studentized residuals were greater than 3 standard deviations away from the mean residual.

Mane hair length: Trend towards longer hair with the example dietary supplement treatment (P=0.22) over the control. The horses who consumed the example dietary supplement treatment had mane hair that was 8.9% longer at 90 days, compared to those provided the trace minerals in sulfated form.

Shed hair volume: No differences detected in the shed volume. However, it was noted the horses that consumed the example dietary supplement visually appeared to go through the Winter-to-Summer coat transition more rapidly.

No differences were detected for the hoof growth or the body hair growth between the two treatments.

Thus, the same dosage of trace minerals provided in traditional sulfated form and in the amino acid complexes described herein provided improved horse mane growth for those provided the trace minerals in the amino acid complexes. After 90 days the horses had 8.9% longer mane growth when their diet was supplemented with the example dietary formulation when compared to those receiving the same trace mineral dosage via sulfated minerals. No negative impact was assessed as to body hair or hoof growth. Further, the horses that consumed the example dietary supplement were observed to transition between winter and summer coats.

The inventions being thus described, it will be obvious that the same may be varied in many ways. Such variations are not to be regarded as a departure from the spirit and scope of the inventions and all such modifications are intended to be included within the scope of the following claims. The scope of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

1. A method of nutritionally supplementing the diet of a canine, equine or feline comprising:
feeding the canine, equine, or feline a mineral supplement comprising a copper mixed amino acid complex, an iron mixed amino acid complex, a manganese mixed amino acid complex, and a zinc mixed amino acid complex; wherein the zinc complex is in an amount between about 6 wt. % and about 10 wt. %; wherein the iron complex is in an amount between about 2.5 wt. % and about 5 wt. %; wherein the manganese complex is in a concentration of between about 1 wt. % and about 4 wt. %; and wherein the copper complex is in a concentration of between about 0.2 wt. % and about 2 wt. %.

2. The method of claim 1, wherein the zinc complex is in an amount between about 7.5 wt. % and about 9 wt. %; wherein the iron complex is in an amount between about 3 wt. % and about 4 wt. %; wherein the manganese complex is in a concentration of between about 1.5 wt. % and about 2.5 wt. %; and wherein the copper complex is in a concentration of between about 0.3 wt. % and about 1 wt. %.

3. The method of claim 1, wherein the mineral supplement comprises the zinc, iron, manganese and copper complexes in a ratio between about 8:3:2:0.5 to about 10:4:3:1.

4. The method of claim 1, wherein the feeding comprises providing a pet food comprising the mineral supplement; wherein the ratio of the mineral supplement to the pet food on a weight basis is between about 1:200 and about 1:1300.

5. A method of reducing shedding in a canine, equine or feline comprising:
feeding the canine, equine or feline a mineral supplement comprising a copper mixed amino acid complex, an iron mixed amino acid complex, a manganese mixed amino acid complex, and a zinc mixed amino acid complex; wherein the zinc complex is in an amount between about 6 wt. % and about 10 wt. %; wherein the iron complex is in an amount between about 2.5 wt. % and about 5 wt. %; wherein the manganese complex is in a concentration of between about 1 wt. % and about 4 wt. %; and wherein the copper complex is in a concentration of between about 0.2 wt. % and about 2 wt. %.

6. The method of claim 5, wherein the zinc complex is in an amount between about 7.5 wt. % and about 9 wt. %; wherein the iron complex is in an amount between about 3 wt. % and about 4 wt. %; wherein the manganese complex is in a concentration of between about 1.5 wt. % and about 2.5 wt. %; and wherein the copper complex is in a concentration of between about 0.3 wt. % and about 1 wt. %.

7. The method of claim 5, wherein the mineral supplement comprises the zinc, iron, manganese and copper complexes in a ratio between about 8:3:2:0.5 to about 10:4:3:1.

8. The method of claim 5, wherein the feeding comprises providing a pet food comprising the mineral supplement; wherein the ratio of the mineral supplement to the pet food on a weight basis is between about 1:200 and about 1:1300.

9. The method of claim 8, wherein the mineral supplement improves the canine or the feline coat color and shine, or the equine mane length.

10. A method of manufacturing a pet food for a canine, equine or a feline comprising:
adding a mineral supplement to the pet food; wherein the mineral supplement comprises a copper mixed amino acid complex, an iron mixed amino acid complex, a manganese mixed amino acid complex, and a zinc mixed amino acid complex;
wherein the zinc complex is in an amount between about 6 wt. % and about 10 wt. % of the mineral supplement; wherein the iron complex is in an amount between about 2.5 wt. % and about 5 wt. % of the mineral supplement; wherein the manganese complex is in a concentration of between about 1 wt. % and about 4 wt. % of the mineral supplement; and wherein the copper complex is in a concentration of between about 0.2 wt. % and about 2 wt. % of the mineral supplement.

11. The method of claim 10, wherein the mineral supplement comprises the zinc, iron, manganese and copper complexes in a ratio between about 8:3:2:0.5 to about 10:4:3:1.

12. The method of claim 10, wherein the mineral supplement is added to the pet food at a weight ratio of between about 1:200 and about 1:1300 of the mineral supplement to the pet food.

13. The method of claim 12, wherein the pet food comprises one or more of chicken, lamb, beef, pork, salmon, corn, a grain, a probiotic, a starch, a vegetable, a fruit, an animal fat, a seed oil, and a vegetable oil.

14. The method of claim 13, wherein the pet food is kibble.

15. A method of reducing shedding in a canine or feline comprising:
feeding the canine or feline a mineral supplement comprising a copper amino acid complex, an iron amino acid complex, a manganese amino acid complex, and a zinc amino acid complex; wherein the zinc complex is in an amount between about 3 wt. % and about 8 wt. %; wherein the iron complex is in an amount between about 1 wt. % and about 4 wt. %; wherein the manganese complex is in a concentration of between about 0.5 wt. % and about 3 wt. %; and wherein the copper complex is in a concentration of between about 0.1 wt. % and about 2 wt. %.

16. The method of claim 15, wherein the mineral supplement comprises the zinc, iron, manganese and copper complexes in a ratio between about 5:2:1:0.3 to about 8:3:2:1.

17. The method of claim 15, wherein the feeding comprises providing a pet food comprising the mineral supplement; wherein the ratio of the mineral supplement to the pet food on a weight basis is between about 1:200 and about 1:1300.

18. The method of claim 17, wherein the mineral supplement improves the canine or the feline coat color and shine.

19. The method of claim 1, wherein the mixed amino acid complex comprises lysine and glutamic acid in a ratio of from about 2:1 to about 1:2.

20. The method of claim 19, wherein the ratio of lysine to glutamic acid is about 1:1.
